(19) United States
(12) Patent Application Publication
(10) Pub. No.: US 2006/0019376 A1
(43) Pub. Date: Jan. 26, 2006

(54) Fermentation chamber and mixing apparatus

(76) Inventors: Henry R. Bungay III, Troy, NY (US); James S. Bungay, Troy, NY (US); John G. Sigsby, Glenmont, NY (US)

Correspondence Address: Hoffman, Warnick & D'Alessandro LLC, 14th Floor, 75 State Street, Albany, NY, 12207, US

(21) Appl. No.: 11/186,610

(22) Filed: Jul. 21, 2005

Related U.S. Application Data
(60) Provisional application No. 60/589,942, filed on Jul. 21, 2004.

(51) Int. Cl. C12M 1/02

(52) U.S. Cl. 435/303.3; 435/304.2; 435/809

(57) ABSTRACT

The claimed invention provides a fermentation chamber that is inexpensive to produce and use and is capable of providing sufficient agitation of the liquid medium to ensure proper mixing and/or aeration without damaging delicate components. The invention further provides a mixing apparatus for agitating one or more such fermentation chambers. In a first embodiment, the claimed invention provides a fermentation chamber, comprising: a rigid top portion; a rigid bottom portion; and a flexible member connecting the rigid top portion and the rigid bottom portion.

### CROSS-REFERENCE TO RELATED APPLICATIONS

This application claims the benefit of co-pending U.S. Provisional Application No. 60/589,942, filed Jul. 21, 2004, which is hereby incorporated herein.

### BACKGROUND OF THE INVENTION

(1) Technical Field

The present invention relates generally to the field of fermentation devices and more specifically to a fermentation chamber and mixing apparatus.

(2) Description of Related Art

The development and commercialization of many processes in the fields of medicine, chemistry, and agriculture require the use of fermentation devices or “bioreactors.” Cell culturing, for example, is often carried out in vessels that permit the mixing of cells with nutritive media and oxygen. In industrial applications, such processes are often carried out in very large vessels, often greater than 50 liters in capacity. During research and development, however, it is generally desirable to test such processes on a much smaller scale. Historically, fermentation devices and bioreactors with volumes of 50 liters or less have suffered from various deficiencies.

Many problems with existing devices lie with the mixing mechanisms employed. Some processes use enzymes immobilized on the surfaces of particles within a liquid medium. As a result, most of the enzymatic activity is limited to the surfaces of the particles. Any method of mixing the liquid medium that causes abrasion of the particles will necessarily reduce enzymatic activity. Similar damage can be caused to cells or microorganisms within a liquid medium.

Magnetic stirrers, for example, are inapplicable to some processes, including the culturing of cells or microorganisms, due to the tendency of the magnetic stirrer, which necessarily contacts an interior surface of the vessel, to damage delicate components, such as living cells and microorganisms, that become trapped between the magnetic stirrer and the vessel wall. Attempts have been made to alleviate this disadvantage through the use of superconductive materials. TC Tech Corp. (www.tc-tech.com), for example, markets a mixing device wherein a disposable impeller is levitated above the vessel's bottom, thereby eliminating the potential for entrapment of cells or microorganisms between the impeller and the vessel wall. Such devices are, however, expensive to use, due to their need to operate at superconducting temperatures.

U.S. Patent Application Publication No. 2003/0008389 to Carll describes a disposable cell culture vessel with a hollow sleeve in its interior, into which is placed a magnetic stirrer. In some embodiments, the sleeve is fitted with a flexible blade. Such a device also reduces or eliminates the tendency of magnetic mixers to damage delicate components. However, due in part to the fact that the mixing action of the device is provided by the simple rotation of a magnetic bar, the device is incapable of providing greater agitation or aeration of the liquid medium. Rather, the placement of a magnet within the hollow sleeve “allows the gentle rotation of the impeller and the subsequent undulation of the flexible blades when an adjustable magnetic force, such as a magnetic stir plate, is applied to the vessel. This creates a gentle stirring of the cells, which keeps the cells in suspension and prevents the cells from shearing.” ¶ 17. Where more vigorous agitation or greater aeration of the liquid medium is needed, such a device is inadequate.

Other devices utilize blades or similar mechanisms to mix their liquid contents. U.S. Pat. No. 3,468,520 to Duryea et al., for example, describes a paddle-like mechanism residing within a bottle, which is designed to agitate a suspension of cells. Such devices, however, require the introduction of a foreign object, in the form of the mixing mechanism, into the liquid medium. This greatly increases the possibility of contamination of the medium by substances or organisms residing on the mixing mechanism. Avoidance of such contamination requires thorough cleaning and sterilization of the mixing mechanism before each use, which can greatly increase not only the burden and expense of using such devices, but also the level of technical experience required by its users.

Others have attempted to integrate the mixing mechanism into the vessel itself. U.S. Pat. No. 3,432,149 to Stalberg et al., for example, describes an apparatus for stirring a liquid having internal wings, wherein rotation of the device along its longitudinal axis exerts a dragging action on the liquid. However, such a device is capable of exerting a dragging action on only a small portion of the liquid. “The height of the liquid-dragging part of the vessel is at the most half of the intended liquid level, suitably no more than one-third thereof and preferably about one-fourth thereof.” Col. 2, lines 43-46. In addition, such a device is incapable of aerating the liquid by, for example, projecting a portion of the liquid above the level of the standing liquid, thereby creating turbulence between the surface of the liquid and a gaseous layer above it.

Attempts have been made to eliminate the need for internal mixing mechanisms altogether. U.S. Pat. No. 4,373,029 to Nees, and U.S. Pat. No. 3,540,700 to Freedman et al., for example, describe devices for pivotally rotating vessels containing cells and a nutrient medium. There is a limit, however, to the degree of mixing attainable with such devices. For example, Nees notes that “acceleration magnitudes are essentially determined only by the gravity of the microcarrier in the earth's gravitational field, reduced by the viscosity of the nutrient solution.” Col. 2, lines 11-14. Thus, for processes requiring a greater degree of mixing or agitation, including, for example, processes requiring greater aeration of the liquid medium, such devices are not useful.

A need exists, therefore, for a device that avoids the above limitations. Specifically, there is a need for a fermentation chamber and mixing apparatus that (1) will not damage delicate components of the liquid medium, such as living cells and microorganisms, (2) can provide sufficient agitation of the liquid medium to ensure proper mixing and/or aeration, (3) is inexpensive to produce and use.

### SUMMARY OF THE INVENTION

The claimed invention provides a fermentation chamber that is inexpensive to produce and use and is capable of providing sufficient agitation of the liquid medium to ensure proper mixing and/or aeration without damaging delicate components. The invention further provides a mixing apparatus for agitating one or more such fermentation chambers. When used in processes utilizing particle-immobilized enzymes, the claimed invention permits adjustment of the degree of agitation of the liquid medium to ensure movement over surfaces of the particles with little or no grinding of the particles against each other.

The chamber of the claimed invention is applicable to a wide variety of processes, including the culturing of living cells and microorganisms. In a first embodiment, the claimed invention provides a fermentation chamber comprising: a rigid top portion; a rigid bottom portion; and a flexible member connecting the rigid top portion and the rigid bottom portion.

In a second embodiment, the claimed invention provides a fermentation chamber comprising: a rigid bottom portion; at least one arm; and at least one pin adjacent the at least one arm for attaching the fermentation chamber to a mixing apparatus.

In a third embodiment, the claimed invention provides a mixing apparatus comprising: a drive mechanism; a first mixing bar; a second mixing bar; and a fermentation chamber having: a rigid bottom portion; at least one arm; and at least one pin adjacent the at least one arm for attaching the fermentation chamber to a mixing bar.

In a fourth embodiment, the claimed invention provides a fermentation chamber comprising: a rigid top portion; a flexible bottom portion; and a receptacle for the flexible bottom portion.

In a fifth embodiment, the claimed invention provides a fermentation chamber comprising: a flexible top portion; a flexible bottom portion; and a receptacle for at least one of the flexible top portion and the flexible bottom portion.

The foregoing and other features of the invention will be apparent from the following more particular description of embodiments of the invention.

### BRIEF DESCRIPTION OF THE DRAWINGS

The embodiments of this invention will be described in detail, with reference to the following figures, wherein like designations denote like elements, and wherein:

FIG. 1 shows a side elevational view of one embodiment of the invention, wherein the chamber comprises a rigid top portion and a rigid bottom portion connected by a flexible member.

FIG. 2 shows a cross-sectional view of a rigid bottom portion of the claimed invention with internal projections formed by different methods.

FIG. 3 shows a side elevational view of an alternative embodiment of the rigid top portion of the claimed invention with elements for adding materials to or removing materials from the chamber.

FIGS. 4A and 4B show a side elevational view and cross-sectional view, respectively, of an alternative embodiment of the claimed invention.

FIG. 5 shows a side view of an alternative embodiment of a fermentation chamber according to the present invention.

FIG. 6 shows a mixing apparatus for use with fermentation chambers of the present invention.

### DETAILED DESCRIPTION OF THE INVENTION

Reference will now be made in detail to the preferred embodiments of the claimed invention, several examples of which are illustrated in the accompanying drawings. Additional information can be found in Appendices A and B, which are herein incorporated by reference.

As used herein, the term “fermentation” means a process for the production of a product by culturing cells or microorganisms, the process of culturing cells or microorganisms, or a process for the bioconversion of one material to another.

The term “chamber” means a container capable of holding a particular liquid medium. In addition, when sealed, said chamber is capable of holding a particular gaseous medium.

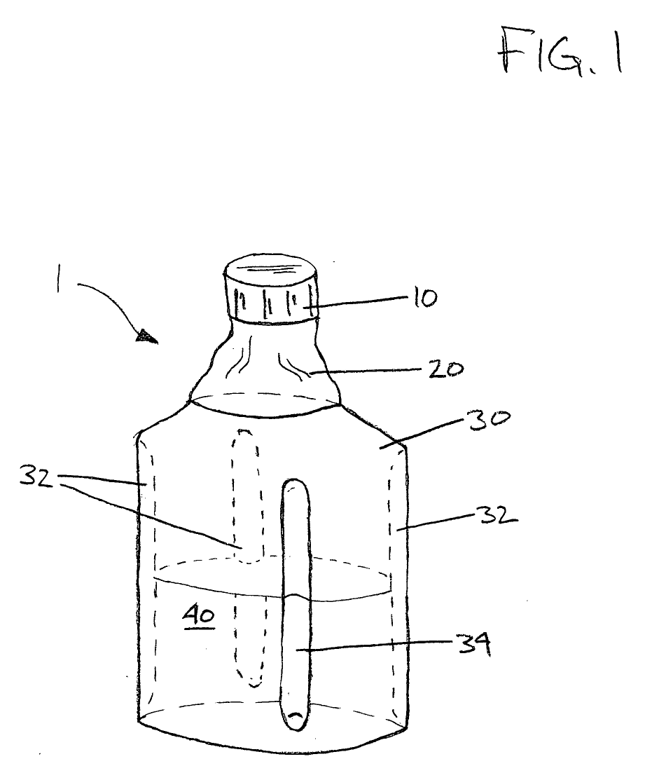

Referring to FIG. 1, one embodiment of a fermentation chamber 1 of the claimed invention is shown. Top portion 10 and bottom portion 30 are of a rigid material and connected by flexible member 20. Top portion 10 and bottom portion 30 may be of any rigid material known in the art, including, for example, a metal, a ceramic, a glass, polyethylene, polystyrene, a polyester, or polypropylene. Flexible member 20 may be of any flexible material known in the art, including, for example, polyethylene, a polyester, polypropylene, silicon, or rubber. Flexible member 20 may be joined to top portion 10 and bottom portion 30 by any number of means, including, for example, adhesives, clamps, stitching, threaded members, and thermal attachment (i.e., melting a portion of one or both components at a point where they are to be joined). Preferably, the attachment of flexible member 20 to top portion 10 and bottom portion 30 permits the easy sealing and unsealing of chamber 1, where, for example, top portion 10 is a cap which threads onto flexible member 20.

Bottom portion 30 may be provided with at least one projection 32 on its inner surface. Although projections 32 are shown in a vertical orientation along the inner vertical walls of bottom portion 30, it should be noted that such projections need not be orientated vertically and may be positioned on any inner surface of bottom portion 30, including its horizontal bottom surface. As depicted in FIG. 1, projections 32 correspond to indentations 34 in an outer surface of bottom portion 30. Such projections and indentations can be found in what are commonly called “blow molded” bottles, and are generally composed of a glass or plastic material.

When chamber 1 is rotated back-and-forth about its longitudinal axis, liquid 40 naturally rotates at a slower rate than chamber 1, due to fluid inertia. Projections 32 obstruct the movement of liquid 40, and particularly so upon the periodic reversal of the direction of rotation of chamber 1. This obstruction of the movement of liquid 40 results in its mixing and aeration. The back-and-forth rotation of chamber 1 may be provided by any means known in the art, including, for example, an eccentric or piston drive.

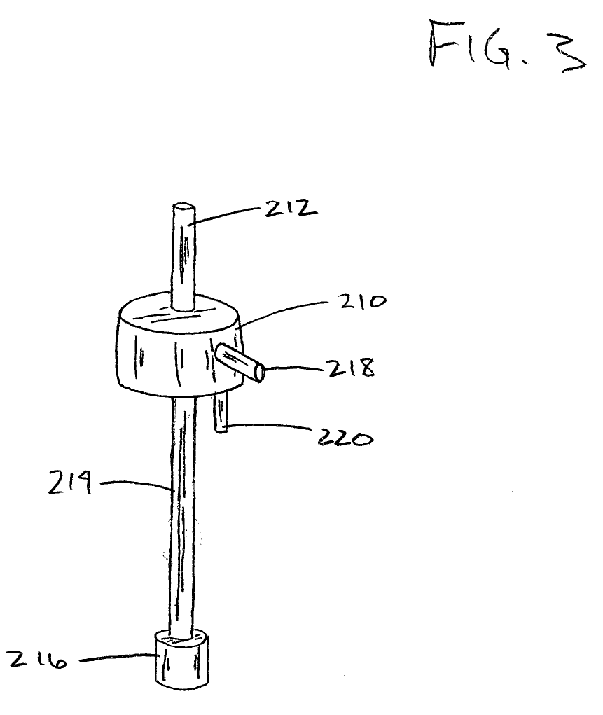

In addition, the complete or substantial immobilization of top portion 10 increases the mixing and/or aeration of liquid 40. Top portion 10 may be immobilized by any number of means, including, for example, clamps, brackets, and rods. Such immobilization also permits easier sampling or monitoring of the contents of chamber 1, as will be described in detail below with reference to FIG. 3.

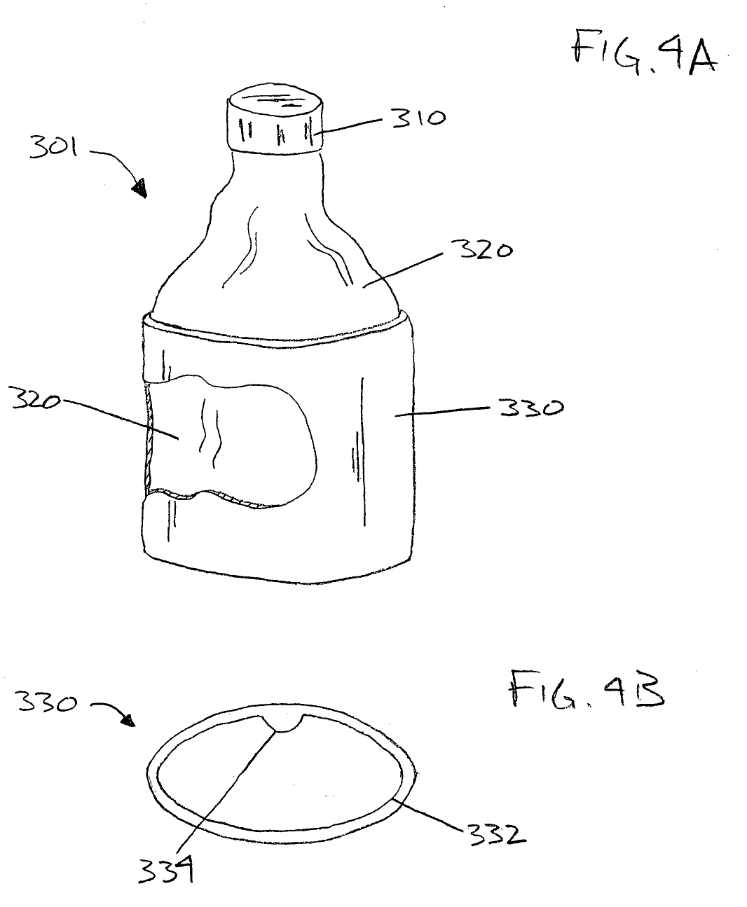

In another embodiment of the invention, the need for projections 32 on an inner surface of bottom portion 30 is obviated by the non-circular cross-sectional shape of bottom portion 30. The non-circular shape of bottom portion 30 results in obstruction of the movement of liquid 40, providing mixing and/or aeration of liquid 40. Many non-circular shapes are capable of obstructing the movement of liquid 40, although elliptical and rectangular shapes are particularly useful. An example of a suitable cross-sectional shape is depicted in FIG. 4B, described below with reference to a third embodiment of the invention.

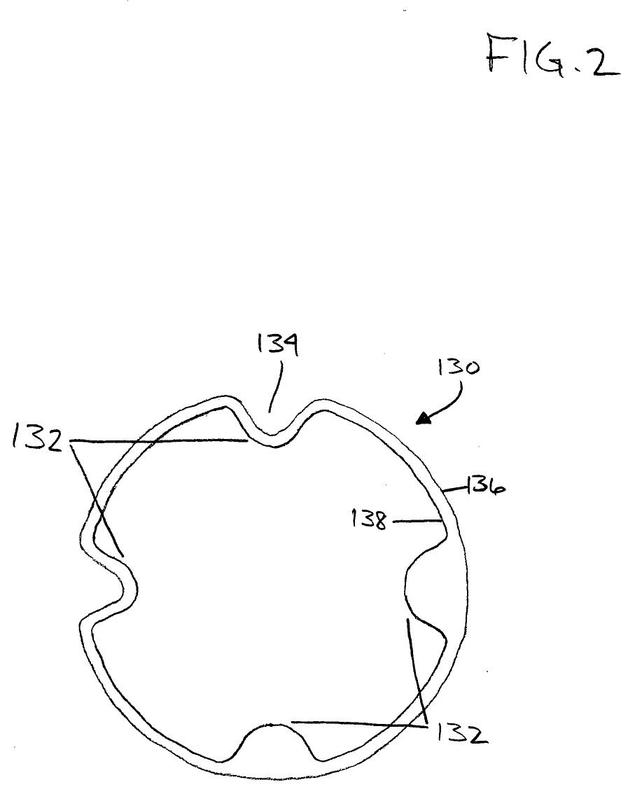

FIG. 2 shows a cross-sectional view of bottom portion 130. As shown, projections 132 on an inner wall 138 may be produced by the formation of an indentation 134 on an outer wall 136 or by the formation or attachment of additional material to an inner wall 138. Such additional material may be part of the original molding of bottom portion 130, where bottom portion 130 is a molded product. Alternatively, such additional material may be attached to an inner wall 138 of bottom portion 130 by any means known in the art, including, for example, adhesives, screws, bolts, friction connections, and thermal attachment.

Referring now to FIG. 3, an alternative embodiment of top portion 210 is shown with optional additional elements. First external element 212 and first internal element 214 comprise interconnected hollow vessels for the introduction of materials to or the removal of materials from the interior of the chamber. Optionally, first internal element 216 may terminate in a submersible element 216, which may comprise any number of devices, including, for example, a sensor, a heating element, a cooling element, and a diffuser for the introduction of gaseous bubbles into liquid 40. Sensors include those known in the art, including, for example, pH electrodes, thermometers, turbidity probes, or dissolved oxygen electrodes. Where submersible element 216 is a sensor, a heating element, a cooling element, or similar device, materials need not necessarily be introduced to or removed from the chamber. Rather, first external element 212 and first internal element 214 may provide a pathway to submersible member 216, for the provision of device components, such as electrical wires or heating and cooling fluids.

Optionally, top portion 210 may include, in addition to or in place of the elements described above, second external element 218 and second internal element 220, also interconnected hollow vessels. Unlike first internal element 214, which ideally descends into the liquid contents of the chamber, second internal element 220 terminates at a point above the standing level of liquid in the chamber. Second external element 218 and second internal element 220, therefore, may be used to add materials to the chamber or to remove gaseous materials from the chamber. Optionally, second internal element 220 may terminate in a device such as submersible element 216, which is used to monitor or alter a physical or chemical property of the gaseous contents of the chamber. Any of the optional elements described above may be of a material or combination of materials known in the art, including, for example, glass, metal, polyethylene, polypropylene, a polyester, silicon, or rubber.

Referring now to FIGS. 4A and 4B, an alternative embodiment of the claimed invention is shown, wherein chamber 301 is comprised of rigid top portion 310 and flexible bottom portion 320. Thus, a substantial portion of chamber 301 is comprised of a flexible bag-like structure. As such, bottom portion 320 is capable of adapting its shape in response to the volume of its fluid contents, the shape of an external body, or both. Receptacle 330, for example, is a hollow non-circular member capable of supporting bottom portion 320. The non-circular shape of receptacle 330 results in obstruction of the movement of the liquid contents of chamber 301, obviating the need for internal projections. Such projections may optionally be included, either on an inner surface of bottom portion 320 or on an inner surface 332 of receptacle 330. In the latter embodiment, bottom portion 320 will adapt its shape to conform to projection 334. Optionally, both the top portion and the bottom portion of the chamber may be composed of flexible materials.

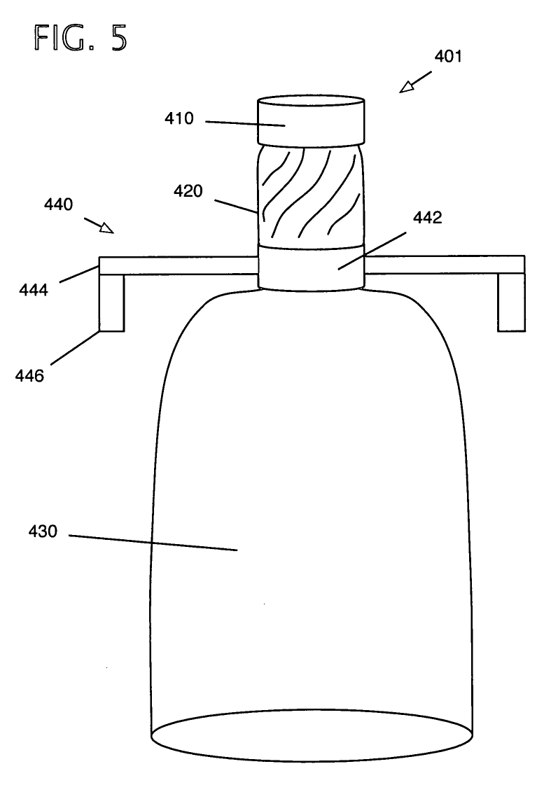

Referring to FIG. 5, an alternative embodiment of a fermentation chamber according to the present invention is shown, wherein chamber 401 is comprised of rigid top portion 410, rigid bottom portion 430, flexible member 420, and cap member 440. Preferably, cap member 440 includes a cap 442 adapted to threadably engage a threaded neck (not shown) of rigid bottom portion 430. As such, rigid bottom portion 430 may be disposable or recyclable while rigid top portion 410, flexible member 420, and cap member 440 may be reusable. Rigid top portion 410, flexible member 420, and cap member 440 preferably comprise an integrated unit.

As described above, rigid cap member 410 may include one or more apertures (not shown) to facilitate the sampling, monitoring, etc. of the contents of chamber 401 or the introduction of materials to chamber 401. Of course, where sampling, monitoring, etc. of chamber contents is unnecessary, an alternative embodiment my include only rigid bottom portion 430 and cap member 440. That is, flexible member 420 and rigid top portion 410 are unnecessary if entry into an interior of rigid bottom portion 430 is not required. In such an embodiment, cap 442 would preferably include a closed or closable top.

Cap member 440 comprises cap 442, one or more laterally extending arms 444, and one or more pins 446 extending from each arm 444. Such an arrangement of arms 444 and pins 446 permits chamber 401 to be mixed via a mixing apparatus, which will be described below.

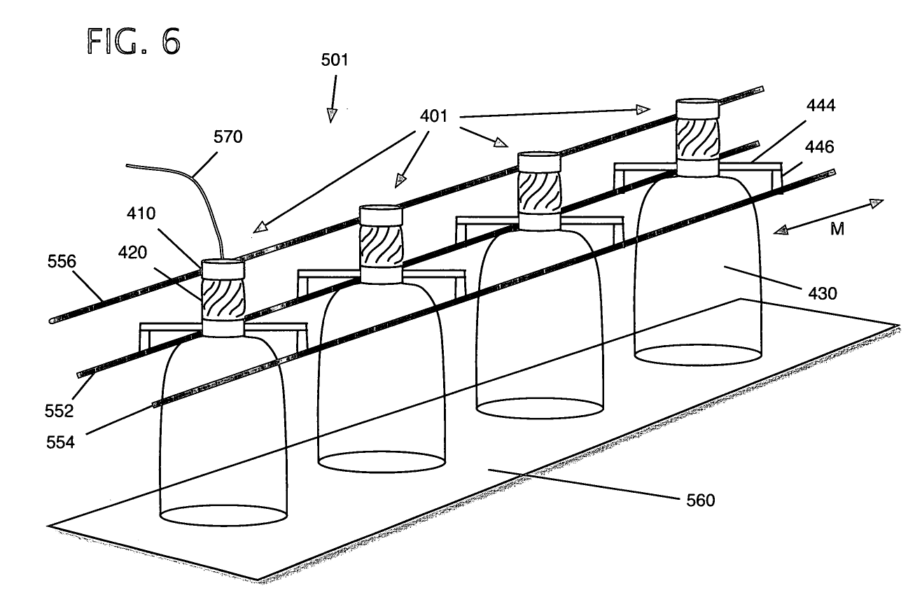

Referring now to FIG. 6, a mixing apparatus 501 is shown for providing an agitating motion to one or more fermentation chambers 401. Mixing apparatus 501 comprises mixing bars 552, 554 and, optionally, a stabilizing bar 556. Mixing apparatus 501 may further comprise a drip pan 560 for collecting any liquids that may escape from chambers 401 during mixing, sampling, monitoring, etc.

One or more fermentation chambers 401 are arranged between mixing bars 552, 554. Preferably, each chamber has a form similar to chamber 401 as shown in FIG. 5, such that arms 444 and pins 446 align with mixing bars 552, 554. Alternatively, arms 444 and pins 446 may be included on rigid bottom portion 430 rather than cap 442. Of course, chambers having other forms, such as those shown in FIGS. 1 and 4A, may be adapted for use with mixing apparatus 501. For example, one or more arms 444 and pins 446 may be incorporated into or secured to bottom portion 30 (FIG. 1) or receptacle 330 (FIG. 4A) using a clamp, adhesive, etc.

Arms 444, pins 446, and/or mixing bars 552, 554 may include any number of apparatuses (not shown) for securing chambers 401 to mixing bars 552, 554. One suitable apparatus includes holes or slots in mixing bars 552, 554 adapted to receive pins 446, although other apparatuses are possible, as would be known to one of ordinary skill in the art.

A drive mechanism (not shown) provides a back and forth motion M to one or more arms 444. Any known or later developed drive mechanism may be used, including, for example, an eccentric drive, a piston drive, etc. In one embodiment, first mixing bar 552 is immobile while second mixing bar 554 is connected to a drive mechanism. The back and forth motion M of second mixing bar 554 provides agitation to chambers 401 and their contents. In an alternative embodiment, both first mixing bar 552 and second mixing bar 554 are connected to a drive mechanism, which provides back and forth motion M to each. In such an embodiment, the relative degree and/or speed of back and forth motion M may be reduced as compared to an embodiment wherein only second mixing bar 554 is agitated.

In a situation where contents of chamber 401 will be monitored, sampled, etc., mixing apparatus 501 may further comprise a stabilizing bar 556 to which the rigid top portion 410 of each chamber 401 may be secured. Such an arrangement permits the connection and/or insertion of a line 570 to rigid top portion 410, whereby flexible portion 420 permits the agitation of rigid bottom portion 430 without agitation of rigid top portion 410. Line 570 may include any number of apparatuses, including wires, tubing, etc., as described above. Rigid top portion 410 may be secured to stabilizing bar 556 by any known or later developed methods, including, for example, clamps, rings, straps, screws, bolts, magnets, hooks and loops, etc.

While this invention has been described in conjunction with the specific embodiments outlined above, it is evident that many alternatives, modifications, and variations will be apparent to those skilled in the art. Accordingly, the embodiments of the invention as set forth above are intended to be illustrative, not limiting. Various changes may be made without departing from the spirit and scope of the invention as defined in the following claims.

What is claimed is:

1. A fermentation chamber comprising:
a rigid top portion;
a rigid bottom portion; and
a flexible member connecting the rigid top portion and the rigid bottom portion.

2. The fermentation chamber of claim 1, wherein the rigid top portion and the rigid bottom portion include at least one material selected from a group consisting of a metal, a glass, a ceramic, a polyester, polyethylene, polypropylene, and polystyrene.

3. The fermentation chamber of claim 1, wherein the flexible member includes at least one material selected from a group consisting of a rubber, a silicone, a polyester, polyethylene, and polypropylene.

4. The fermentation chamber of claim 1, further comprising at least one projection residing on an inner surface of the rigid bottom portion and capable of obstructing the movement of liquids within the chamber.

5. The fermentation chamber of claim 1, wherein the rigid top portion includes at least one aperture for receiving at least one hollow vessel extending from the exterior of the chamber to the interior of the chamber.

6. The fermentation chamber of claim 5, wherein a portion of the at least one hollow vessel extending into the interior of the chamber includes at least one device selected from a group consisting of a sensor, a heating device, a cooling device, a sampling device, and a diffusing device.

7. The fermentation chamber of claim 1, wherein the rigid bottom portion is substantially non-circular in cross section.

8. The fermentation chamber of claim 7, wherein the rigid bottom portion has an elliptical cross section.

9. The fermentation chamber of claim 1, further comprising a cap member adjacent the flexible member having:
a cap;
at least one arm; and
at least one pin adjacent the at least one arm for attaching the fermentation chamber to a mixing apparatus.

10. A fermentation chamber comprising:
a rigid bottom portion;
at least one arm; and
at least one pin adjacent the at least one arm for attaching the fermentation chamber to a mixing apparatus.

11. The fermentation chamber of claim 10, wherein the at least one arm is attached to a cap.

12. The fermentation chamber of claim 10, wherein the rigid bottom portion includes at least one material selected from a group consisting of a metal, a glass, a ceramic, a polyester, polyethylene, polypropylene, and polystyrene.

13. The fermentation chamber of claim 10, further comprising at least one projection residing on an inner surface of the rigid bottom portion and capable of obstructing the movement of liquids within the chamber.

14. The fermentation chamber of claim 10, wherein the rigid bottom portion is substantially non-circular in cross section.

15. The fermentation chamber of claim 14, wherein the rigid bottom portion has an elliptical cross section.

16. A mixing apparatus comprising:
a drive mechanism;
a first mixing bar;
a second mixing bar; and
at least one fermentation chamber having:
a rigid bottom portion;
at least one arm; and
at least one pin adjacent the at least one arm for attaching the fermentation chamber to a mixing bar.

17. The mixing apparatus of claim 16, wherein the drive mechanism includes at least one of an eccentric drive and a piston drive.

18. The mixing apparatus of claim 16, wherein the drive mechanism moves one of the first mixing bar and the second mixing bar in a back and forth motion.

19. The mixing apparatus of claim 16, wherein the at least one arm is located on a surface of a cap.

20. The mixing apparatus of claim 16, wherein the rigid bottom portion has an elliptical cross section.

21. The mixing apparatus of claim 16, further comprising a drip pan.

22. A fermentation chamber comprising:
a rigid top portion;
a flexible bottom portion; and
a receptacle for the flexible bottom portion.

23. The fermentation chamber of claim 22, wherein the rigid top portion and the rigid bottom portion include at least one material selected from a group consisting of a metal, a glass, a ceramic, a polyester, polyethylene, polypropylene, and polystyrene.

24. The fermentation chamber of claim 22, wherein the flexible bottom portion includes at least one material selected from a group consisting of a rubber, a silicone, a polyester, polyethylene, and polypropylene.

25. The fermentation chamber of claim 22, wherein at least one of the flexible bottom portion and the receptacle includes at least one projection residing on an inner surface and which is capable of obstructing the movement of liquids within the chamber.

26. The fermentation chamber of claim 22, wherein the rigid top portion includes at least one hollow vessel extending from the exterior of the chamber to the interior of the chamber.

27. The fermentation chamber of claim 26, wherein a portion of the at least one hollow vessel extending into the interior of the chamber includes at least one device selected from a group consisting of a sensor, a heating device, a cooling device, a sampling device, and a diffusing device.

28. The fermentation chamber of claim 22, wherein at least one of the flexible bottom portion and the receptacle is substantially non-circular in cross section.

29. A fermentation chamber comprising:
a flexible top portion;
a flexible bottom portion; and
a receptacle for at least one of the flexible top portion and the flexible bottom portion.

30. The fermentation chamber of claim 29, wherein the flexible top portion and flexible bottom portion include at least one material selected from a group consisting of a rubber, a silicone, a polyester, polyethylene, and polypropylene.

31. The fermentation chamber of claim 29, further comprising at least one obstruction capable of obstructing the movement of liquids located on at least one of the flexible top portion, the flexible bottom portion, and the receptacle.

32. The fermentation chamber of claim 29, wherein at least one of the flexible top portion and the flexible bottom portion includes at least one hollow vessel extending from the exterior of the chamber to the interior of the chamber.

33. The fermentation chamber of claim 32, wherein a portion of the at least one hollow vessel extending from the exterior of the chamber to the interior of the chamber includes at least one device selected from a group consisting of a sensor, a heating device, a cooling device, a sampling device, and a diffusing device.

34. The fermentation chamber of claim 29, wherein the flexible bottom portion is substantially circular in cross section.
